(19) United States
(12) Patent Application Publication
(10) Pub. No.: US 2015/0276764 A1
(43) Pub. Date: Oct. 1, 2015

(54) DETERMINING DISEASE STATES USING BIOMARKER PROFILES

(71) Applicant: SLUPSKY, CAROLYN, Davis, CA (US)

(72) Inventor: Carolyn Slupsky, Davis, CA (US)

(21) Appl. No.: 14/440,869

(22) PCT Filed: Nov. 5, 2013
(86) PCT No.: PCT/US13/68592

§ 371 Date: May 5, 2015

Related U.S. Application Data
(60) Provisional application No. 61/722,724, filed on Nov. 5, 2012.

(51) Int. Cl. G01N 33/68; G06F 19/00; G06F 19/24

(52) U.S. Cl. CPC G01N 33/6893; G06F 19/24; G06F 19/345; G01N 2333/16; G01N 2570/00; G01N 2333/4746

(57) ABSTRACT

A method of determining the state of a disease in a subject has the following steps: obtaining first and second sets of biological samples from subjects known to have first and second states of the disease, each biological sample comprising a plurality of biomarkers, the first and second states being differentiated by a predetermined period of time; for each sample, generating a profile for each state of the disease based on concentrations of biomarkers from a plurality of samples; generating a profile based on a biological sample from a subject having an unknown state of disease; and comparing the profile for the unknown state of disease to the profiles of the first and second states of disease to determine whether the subject has one of the first state of disease or the second state of disease.

### CROSS-REFERENCE TO RELATED APPLICATION

This application claims the benefit of U.S. Provisional Application No. 61/722,724, filed Nov. 5, 2012, the disclosure of which is hereby incorporated by reference herein in its entirety.

### FIELD

The method described herein relates to distinguishing between disease states using biomarker profiles, such as determining HIV recency and may be particularly useful for incidence measurement.

### BACKGROUND

The rate of new infections in a population is an important metric for monitoring spread of a disease as well as for development of optimal strategies for prevention of a disease. For HIV, studies aimed at counting new infections have shown to be costly and time consuming. Most tests for recent infection classify persons as recently infected based on a below-threshold immune response. Complicating these measurements are individuals who remain classified as recently-infected long after they have been infected, those with AIDS state disease, and those with antiretroviral (ARV) treatment. Thus, statistical methods to estimate the false-recent rate or mean duration of recent infection have been developed to take into account the proportion of persons that are not truly recently infected but produce a recent result with the biomarker.

A recent antibody-based biomarker test has been suggested to improve classification of false-recent individuals. This approach promises some improvement over previous methods; however, this publication only reported on false-recent rates for those with AIDS. The false-recent rate for those on ARV treatment, or those who remain classified as “recently-infected” was not reported. Moreover, antibody testing has been shown to be sensitive to how the assays are performed, relying on the skill of the person performing the assay. Thus, while there may be improvement in classification, the use of antibody assays present challenges for determination of true recent HIV infection.

### SUMMARY

Accurate estimates of HIV incidence are essential to fully characterize the epidemic, monitor transmission patterns, prioritize HIV prevention needs, and design and evaluate interventions. However, the biological assays currently used for incidence are often inaccurate due to a short mean duration of recency and false recent misclassification errors.

We have developed a new approach to incidence based on measurement of metabolites in urine or blood. We have shown that infection of a person by a virus causes observable and quantifiable changes in the metabolite concentrations of urine and blood, and that these changes create a pattern that is specific and sensitive to the infection.

Our approach uses NMR spectroscopy of urine or blood samples combined with multivariate statistical analysis and clinical and medical laboratory data to develop reference metabolite profile patterns of different disease states. These reference patterns (the “biomarker”) can then be used for detection of disease from individuals for which disease status is unknown.

We have used this method to develop reference patterns for the detection of active and latent tuberculosis as well as for HIV, and have some preliminary evidence that our method can distinguish recent HIV infection from non-recent HIV infection.

In order to address the shortcomings of current assays, we used specimens from three biofluids including serum, plasma, and urine. We analyzed the specimens to create reference metabolite profiles for recent and non-recent HIV infection from unblinded samples. We then tested the reference profiles against blinded cohorts to determine FRR and MDRI.

Accordingly, there is provided in one aspect, a method of determining the state of a disease in a subject, the method comprising the steps of:

obtaining at least a first set of diseased biological samples from subjects known to have a first state of the disease and a second set of diseased biological samples from subjects known to have a second state of the disease, each biological sample comprising a plurality of biomarkers, wherein the first state of the disease and the second state of the disease are differentiated by a predetermined period of time;

for each sample, measuring the concentration of a set of biomarkers from among the plurality of biomarkers and generating a profile for each state of the disease, each profile comprising the concentrations from a plurality of samples;

obtaining a biological sample from a subject having an unknown state of disease and measuring the concentration of a set of biomarkers from among the plurality of biomarkers and generating a profile for the unknown state of disease;

comparing the profile for the unknown state of disease to at least the profile of the first state of disease and the second state of disease to determine whether the subject has one of the first state of disease or the second state of disease.

The disease may be HIV infection.

The first state of the disease may be a recent infection and the second state of the disease may be a chronic infection. The recent infection may be an infection contracted within a period of less than 6 months and the chronic infection may be an infection contracted in a period of over 12 months.

There may be at least a third state of disease and the third state of the disease may comprise no disease.

The method may further comprise the step of developing a prognosis of the subject based on the comparison of the profiles.

The biological test sample may be one of blood, blood plasma, blood serum, cerebrospinal fluid, bile acid, saliva, synovial fluid, pleural fluid, pericardial fluid, peritoneal fluid, feces, nasal fluid, ocular fluid, intracellular fluid, intercellular fluid, lymph fluid, and urine.

Comparing the profile may comprise using multivariate statistical analysis. The multivariate statistical analysis may be selected from the group consisting of principal component analysis, discriminant analysis, principal component analysis with discriminant analysis, partial least squares, partial least squares with discriminant analysis, canonical correlation, kernel principal component analysis, non-linear principal component analysis, factor analysis, multidimensional scaling, and cluster analysis.

Each profile may comprise information derived from a respective region of a score plot using multivariate statistical analysis.

The respective concentration of each of the identified metabolites may be determined using a spectrometric technique, wherein the spectrometric technique is any one of liquid chromatography, gas chromatography, liquid chromatography—mass spectrometry, gas chromatography—mass spectrometry, high performance liquid chromatography—mass spectrometry, capillary electrophoresis—mass spectrometry, nuclear magnetic resonance spectrometry (NMR), raman spectroscopy, and infrared spectroscopy.

According to another aspect, there is provided a method of determining the state of a disease in a subject, the method comprising the steps of:

obtaining at least a first set of diseased biological samples from subjects known to have a first state of the disease and a second set of diseased biological samples from subjects known to have a second state of the disease, each biological sample comprising a plurality of discretely identified biomarkers, wherein the first state of the disease and the second state of the disease are differentiated by a predetermined period of time and;

for each sample, measuring the respective concentration of a set of biomarkers from among the plurality of biomarkers and generating a profile for each state of the disease, each profile comprising the concentrations of identified biomarkers from a plurality of samples;

obtaining a biological sample from a subject having an unknown state of disease and measuring the concentration of a set of biomarkers from among the plurality of biomarkers and generating a profile for the unknown state of disease;

using multivariate statistical analysis, comparing the profile for the unknown state of disease to at least the profile of the first state of disease and the second state of disease to determine a probability the subject has one of the first state of disease or the second state of disease.

According to another aspect, there is provided a method of profiling the recency of a disease, comprising the steps of:

obtaining at least a first set of diseased biological samples from subjects known to have a first state of the disease and a second set of diseased biological samples from subjects known to have a second state of the disease, each biological sample comprising a plurality of biomarkers, wherein the first state of the disease and the second state of the disease are differentiated by a predetermined period of time; and

for each sample, measuring the concentration of a set of biomarkers from among the plurality of biomarkers; and

using multivariate statistical analysis, generating a profile for each state of the disease, each profile comprising the concentrations from a plurality of samples.

According to another aspect, there is provided a method of determining the state of a disease in a subject, the method comprising the steps of:

obtaining a plurality of sets of diseased biological samples from subjects, each subject having a known state of the disease, each biological sample comprising a plurality of biomarkers, wherein each state of the disease being one of aggressive, active, acute, recent, chronic, indolent, non-recent, primary, persistent, remission or subclinical;

for each sample, measuring the concentration of a set of biomarkers from among the plurality of biomarkers and generating a profile for each state of the disease, each profile comprising the concentrations from a plurality of samples from subject having the same state of the disease;

obtaining a biological sample from a subject having an unknown state of disease and measuring the concentration of a set of biomarkers from among the plurality of biomarkers and generating a profile for the unknown state of disease;

comparing the profile for the unknown state of disease to the profile of at least two states of the disease determine whether the subject has one of the states of the disease.

According to another aspect, there is provided an apparatus for determining the state of a disease in a subject, the apparatus comprising a housing, comprising a receptacle for receiving a biological specimen and a sensor in communication with the receptacle, the sensor sensing concentration data of a set of biomarkers from the biological specimen. A processor is provided and is programmed to: determine a first profile based on the concentration data; compare the first profile to at least a first predetermined profile indicative of a first state of the disease and to a second predetermined profile indicative of a second state of the disease data, the first state of the disease and the second state of the disease being differentiated by a predetermined period of time; and generate a probability that the subject has the first state of the disease or the second state of the disease. An interface communicates the probability to the user.

The set of biomarkers may comprises a set of 2, 4, 8, 16, 32, 64, 128, or 256 metabolites.

At least one of the sensor and the processor determines a respective concentration of each of the metabolites.

Each of the aspects discussed above may be applicable to other aspects of the invention.

### DESCRIPTION OF THE DRAWINGS

These and other features will become more apparent from the following description in which reference is made to the appended drawings, the drawings are for the purpose of illustration only and are not intended to be in any way limiting, wherein:

FIG. 1 shows an OPLS-DA scores plot showing two different disease states relative to control based on urinary metabolite concentrations derived from ¹H NMR spectra of control subjects (no tuberculosis (n=100) (grey circles)), versus those with active TB (n=50) (black circles), and those with latent TB (n=18) (open circles);

FIG. 2 shows an OPLS-DA scores plot showing two different disease states relative to each other based on urinary metabolite concentrations derived from ¹H NMR spectra of individuals with active TB (n=50) (black circles), and those with latent TB (n=18) (open circles);

FIG. 3 shows an OPLS-DA scores plot showing two different disease states relative to each other based on urinary metabolite concentrations derived from ¹H NMR spectra of individuals with active TB and those without TB, and the classification of individuals being either in the TB state, non-TB state, TB+HIV state, or non-TB+HIV state;

FIG. 4 shows an OPLS-DA scores plot showing the HIV disease state relative to the non-HIV state based on urinary metabolite concentrations derived from ¹H NMR spectra of individuals with no HIV (blue circles), and those with early and late HIV (red circles), and those with confirmed HIV, predicted (green circles);

FIG. 5A shows an OPLS-DA scores plot based on urinary metabolite concentrations derived from ¹H NMR spectra showing two health states (pneumonia (diseased), and non-pneumonia (normal)), with three individuals progressing from the diseased health state to the normal health state over time;

FIG. 5B shows a PCA scores plot based on urinary metabolite concentrations derived from ¹H NMR spectra showing the trajectory of the health state of mice infected with Streptococcus pneumoniae (red) or sham (sterile culture broth) (green), from healthy, to infection, to clearance of pathogen;

FIG. 5C shows an OPLS-DA scores plot based on urinary metabolite concentrations derived from ¹H NMR spectra showing two health states (cancer, and non-cancer), with an individual presenting with cancer pre-surgery, and the health states upon recovery after surgery, where after surgery samples were taken from 1 month to 1 year post-surgery;

FIG. 6 shows an OPLS-DA scores plot based on urinary metabolite concentrations derived from ¹H NMR spectra of individuals in two cancer states: those with pre-cancerous lesions, and those with malignant, metastatic lesions;

FIG. 7 shows OPLS-DA scores plots based on serum metabolite concentrations derived from ¹H NMR spectra of individuals in two disease states relative to HIV infection (recent (<6 months), and non-recent (>1 year)). These states are compared with the HIV negative health state;

FIG. 8A shows a PLS-DA scores plot based on cerebral spinal fluid metabolite concentrations derived from ¹H NMR spectra of individuals in different metabolic states of rabies disease.

FIG. 8B shows a Markov model representing the disease states in FIG. 8A. Those in late state (state 3) disease progress to death, while those in early (state 1) and mid-states (state 2) of disease can survive if appropriate measures are taken;

FIG. 9 shows how targeted profiling differs from the binning technique. In FIG. 9A, the bin of 0.01 will have a total of four different compounds that contribute to the overall spectral area (betaine, TMAO, taurine and myo-inositol). The concentration of any one of these compounds cannot be determined accurately using binning techniques. Using targeted profiling, which models all NMR spectral peaks in a compound, identification of overlapping compounds may be made using multiple regions of the spectrum, as in, for example, FIG. 9B, where some of the overlapping signals from compounds found in FIG. 9A confirm the presence of those chemicals in that region of the spectrum. In this way, accurate quantification of single-peak compounds such as TMAO in 9A may be accomplished; and

FIG. 10 shows a handheld point of care device that can be used to test specimens from a subject.

### DETAILED DESCRIPTION

The method described below relates to distinguishing between disease states using biomarker profiles. The biomarkers are profiled using known techniques in the field of Metabolomics, such as those described in PCT Application No. PCT/CA2008/000670 (Slupsky et al.) and PCT Application no. PCT/CA2010/001583 (Slupsky), both of which are incorporated herein by reference, and both of which refer to methods for obtaining metabolite profiles from fluid or tissue samples from a subject as well as statistical methods that may be used to compare metabolic profiles.

According to one perspective, rather than comparing a diseased population to a healthy population, the method compares different groups of an infected population. One example that will be discussed below is recent HIV infection vs. chronic HIV infection. A recent infection may be considered to be one that has occurred within the past 6 months, and a chronic infection may be considered to be one that has occurred over 12 months ago. Other time frames may be used, depending on the preferences of the user, the disease, and those that can be distinguished using the techniques described herein. The method may be applied to other disease states as well.

In other words, rather than measuring an immune response, we are measuring the metabolic state of the individual.

It has been found that the method may be used to determine the states of a disease, such as cancer. Determining recency may be contrasted to determining the staging of a disease. While staging is a function of tumor growth and metastasis of disease to other organs, recency is time-based. Significantly, the time associated with tumor growth & metastasis is variable across diseases, patient age, etc. so timing is not a good predictor.

While many current methodologies measure the immune response (or changes in the immune response) by generating antibodies to specific cells or proteins, the presently described method assesses the metabolic state of an individual through measurement of metabolites in either urine or blood. Metabolites are those chemicals (generally less than 1,000 Da) that are involved in cellular reactions for energy production, growth, development, signaling and reproduction, and can be taken up, or released from cells according to cellular needs. These chemicals include sugars, amino acids, organic acids, as well as xenobiotic compounds. Metabolomics (or metabonomics as it is sometimes referred), is dedicated to the study of all metabolites in a cell or system and changes that might result from an internal or external stress such as an infection, disease state, or exposure to a toxin. Metabolic changes can result from changes in the chemical reactions that use these metabolites (i.e. metabolic pathways), or the transporters that take up or release these metabolites. We have shown that infection of a person by a virus or bacterium causes major changes both at the cellular level (the site of infection), and systemically (through the innate immune response). These responses include (but are not limited to) signaling of specific immune cells, signaling of apoptosis, changes in transporters, as well as changes in mitochondrial function and energy production—changes that can be observed as changes in metabolite concentrations at the cellular level, and systemically in the blood or urine.

Examples of metabolites that may be measured include 1,3-dimethylurate, levoglucosan, 1-methylnicotinamide, metabolite 1,2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5 (which may be methylamine), metabolite 6 (which may be methylguanidine), N,N-dimethylglycine, O-acetylcarnitine, pantothenate, propylene glycol, pyroglutamate, pyruvate, quinolinate, serine, succinate, sucrose, metabolite 7 (which may be tartrate), taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, and gluconate, galactose, galactitol, galactonate, lactose, phenylalanine, proline betaine, trimethylamine, butyrate, propionate, isopropanol, mannose, 3-methylxanthine, ethanol, benzoate, glutamate and glycerol. Measurements may be made of a subset of these metabolites, or may include other metabolites, as will be recognized by those skilled in the art.

Staging of a disease is defined as “the determination of distinct phases or periods in the course of a disease, the life history of an organism, or any biological process, or the classification of neoplasms according to the extent of a tumor” (The free dictionary.com). By definition, staging refers to progression of a disease over time. In the context of cancer, staging is defined histologically by the TNM system in which the primary tumor (T) is evaluated on its size and/or extent, the regional lymph node involvement is evaluated by the degree of regional lymph node involvement (N), and metastasis is evaluated on the bases of whether metastasis is present or not (M). Staging is typically done through the use of histology, but can also be accomplished through phenotypic means, such as indicating the stage of Alzheimer's disease based on outward symptoms.

Typically, staging refers to the lifetime of a disease, and the progression in an individual. It does not refer specifically to metabolic or immune system changes. Staging is not the same as the state of the disease. For example, a disease state might be a latent state or a dormant state. In these states, the disease does not confer any outward phenotypic signs of the disease. Yet, an individual may have discreet metabolic and/or immunologic changes occurring within. The disease states may further be described as aggressive, active, acute, recent, chronic, indolent, non recent, primary, persistent, remission or subclinical. A disease may transition from one state to another; for example, acute to chronic to subclinical, or any combination thereof. A disease such as cancer may have states such as indolent, or aggressive among others. States may be further characterized by differences in their metabolism, immune response, biological equilibrium and biological control systems, the effects of which that can be measured through biofluids or tissue metabolomics measurements. An indolent cancer could become aggressive, but may remain indolent. Similarly, a latent state of TB, could become active, an active state could transition to a latent state, and a latent state may remain in a latent state for an indefinite period of time.

For the purposes of this document, latency refers to the time between exposure, and when outward phenotypic symptoms become apparent, such as with rabies infection, where it may take anywhere from less than a week to greater than a year for symptoms to develop after infection. A latent state is that state in which the organism displays no outward phenotypic symptoms such as can be the case with TB infection. Dormancy refers to the ability of a pathogen to become inactive. A dormant state is a state in which the pathogen is not active such is the case that can happen with herpes or HIV infection. An indolent state is a slow growing state. Many forms of cancer exhibit indolent states such as prostate cancer. A chronic state is a disease state marked by a long duration. Neurological diseases, metabolic diseases and cardiovascular diseases are often characterized by a chronic disease state. Acute refers to rapid onset or a short course of a disease that is usually severe. An episode of acute disease results in recovery to a state comparable to the patient's condition of health and activity before the disease, in passage into a chronic phase, or in death. Examples of disease that have acute states include the onset of HIV or a Streptococcus pneumoniae or other type of bacterial or viral infection. Aggressive states are best described as a period of rapid growth of cells (such as cancer cells). An invasive state refers to conditions where invasion into tissue occurs (such as by cancer cells or bacterial cells). A remission state is a state or period during which the symptoms of a disease are abated such as a cancer in remission after treatment. A cured state is that state where the disease is no longer present, but other diseases may be still be present. For example, an individual may have diabetes, and may suffer from an acute S. pneumoniae infection that may be cured with antibiotics, but the diabetes will remain. Each one of these states represents a distinct metabolic and immunologic state that can be measured using metabolomics of biofluids or tissue.

Herein, we use the term disease state to mean the state of the disease at the time of sampling. The disease may or may not progress through stages, and may or may not transition from one state (for example indolent, dormant, or latent) to another (such as aggressive, active, or acute). In this way, the disease would not show an obvious progression. The disease may be subclinical. This term is not to be confused with stages or progression.

Thus, a disease state may be a state of early HIV infection (such as acute HIV infection), or may be preclinical (such as the case with an elite controller), or may be chronic. Another way to describe this, is that the state of tuberculosis infection may be acute, or may be chronic, or may be subclinical or latent, but each of these states do not necessarily follow from the other state as one might suggest with stages and progression. Similarly, with cancer, one may have a preclinical state, an actively growing state, a metastatic state, or a remission state. These states can be quantitatively measured. Again, each state does not have to follow from the other state, and a tumor may be in one state, and spontaneously change to another state. In this way, we measure the state of the disease, and not progression or stage of the disease. For example, staging in cancer is not a well-defined process. As noted in the National Cancer Institute's description of stages (http://www.cancer.gov/cancertopics/factsheet/detection/staging), “For many cancers, TNM combinations correspond to one of five stages. Criteria for stages differ for different types of cancer. For example, bladder cancer T3N0M0 is stage III, whereas colon cancer T3N0M0 is stage II.” Thus staging of a disease, or even within a disease, is somewhat arbitrary, and not necessarily linked to physiological changes, but linked with outward phenotypic changes that are qualitative in nature. We define states as a measureable physiological condition that corresponds to the sum of the metabolism of the host and its associated microbiome that may include the presence of pathogens or viruses, that may be measured in a biofluid or tissue.

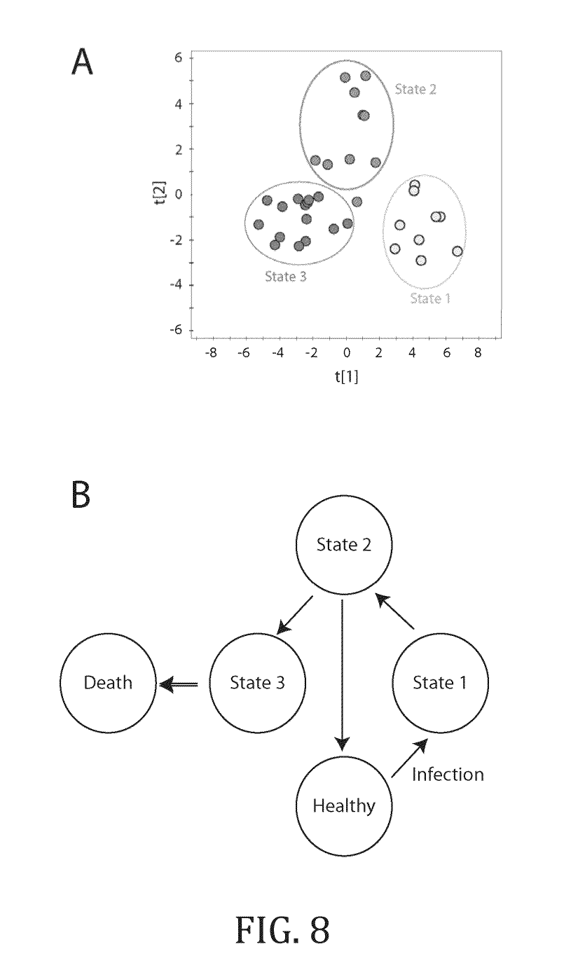

Thus, measuring the progression of a disease state quantitatively is more meaningful than measuring qualitatively, as it does not correspond to an arbitrary stage of disease. Additionally, with detection of a disease state, prognostic indication may be given. For example, in the acute stage of a disease, specific metabolic pathways within the host and the host's microbiome may be altered, and these pathways may cause changes in specific metabolic endproducts that can be measured through the methods of Slupsky. In the case of latent disease, the immune system may be altered, which may cause changes to the gut microbiome, and/or overall metabolism that may be measured through the methods of Slupsky. Latent disease may progress to active disease, but may remain latent or may be cured altogether depending on the health of the individual. In the case of rabies (FIG. 8), three distinct states of metabolic disease were observed, with the first state an early state, corresponding to the first week of hospitalization, that progressed to a middle state (in the second week of hospitalization), that either progressed back to the early state and a state marked by health, or progression to a third state which ultimately resulted in death. This is similar to a description of a Markov tunnel model, where state 2 represents a temporary state that either progresses to death or back to health. Qualitatively, the states of disease are similar, but quantitatively, if intervention is applied prior to evolution to tunnel state, then recovery is possible.

Metabolomics and Infectious Disease

Metabolomics has been successfully applied to studies of host infection including infection by bacteria, parasites, and viruses. Moreover, it has been shown that urinary metabolite profiles may distinguish different bacterial and viral lung infections. Recently, we completed a project on the usefulness of urinary metabolomics for the early diagnosis of Mycobacterium tuberculosis (TB) for third world application. TB is considered difficult to diagnose in its early states because its symptoms resemble other conditions (e.g. influenza, asthma or a general chest infection). Gold standard testing includes sputum smears, nucleic acid amplification followed by bacterial culture to confirm diagnosis.

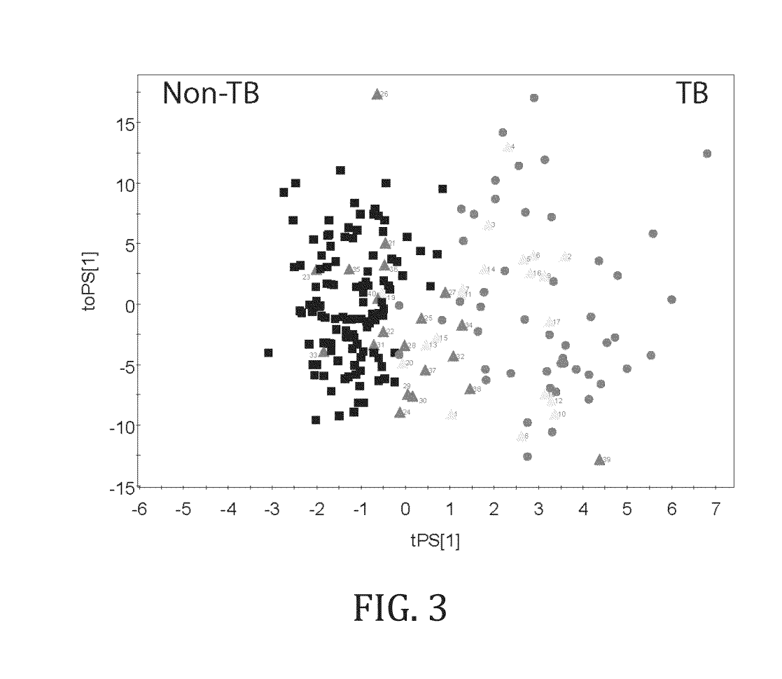

The TB project consisted of two phases. The first phase was an initial comparison of active TB and those without TB (normal) in a North American population with 40 individuals from third world countries including Vietnam, South Africa, and Peru. These samples consisted of urine samples collected from 10 TB−/HIV−; 10 TB−/HIV+; 10 TB+/HIV−; and 10 TB+/HIV+. The second phase of the project was to predict a blinded set of 400 samples that included both TB− and TB+ individuals that may be HIV+ or HIV−. FIG. 3 shows the classification of the phase 1 samples. For those individuals without TB (non-TB), 18/20 were correctly classified as TB−. One individual could not be classified, and one was classified as having TB. Both of these individuals were HIV+. Of those individuals that were determined by other methods to be TB+ (through either culture, sputum smear microscopy, or nucleic acid-based testing), we identified 15 as having TB, with one individual that could be not classified, and four (all HIV+) that were incorrectly classified as normal. On this initial study, we calculated our sensitivity and specificity to be 79% and 95% respectively. Subsequently, we analyzed a blinded set of 400 samples. In this case, our sensitivity and specificity decreased slightly to 65% and 84% respectively. Interestingly, of those samples that were false negatives, 90% could be classified as latent TB.

Significance

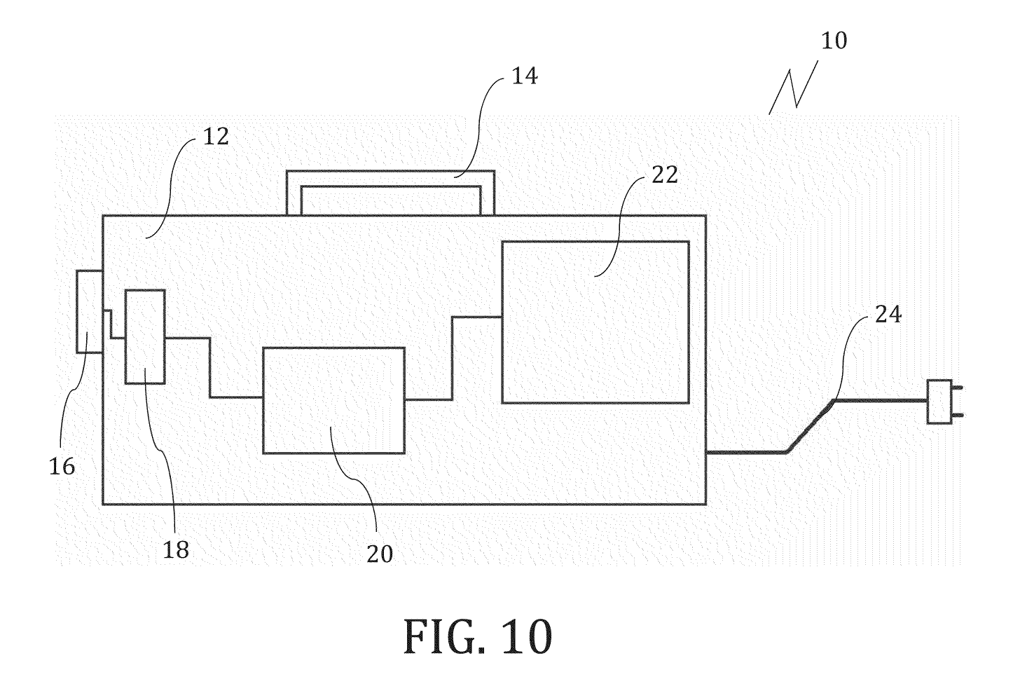

The significance of these findings are:
1. Sensitivity of this test is far greater than testing by sputum smear microscopy, and less than nucleic acid based methods (assuming a sputum sample can be obtained from each patient).
2. When taking into account availability of a suitable sample (i.e. sputum), sensitivity of our test is greater than for nucleic acid based methods since not everyone can provide a suitable sputum sample.
3. Our methods identified infected persons in both first- and third-world settings.
4. Collection of samples is easy, and samples can be collected from everyone (not so with sputum which can only be collected from a fraction of adults and can rarely be collected from children). Although nucleic acid based methods appear to have greater sensitivity, they also rely on obtaining a sputum sample.
5. This test is much faster (results could be turned around within hours) than gold standard (bacterial culture) which takes several weeks.
6. Samples can be collected by minimally trained staff, and data collected in a central testing center only needing one moderately trained individual.
7. With technological advances, and the race to develop a handheld instrument by several companies, a point-of-care device may be developed. Such a device is described below with reference to FIG. 10. In this case, the cost to run per sample will be a fraction of what the cost is today.

These results indicate that metabolomics is effective as a screening diagnostic for acute or active infectious disease. Moreover, our published studies indicate that the metabolic response for each infection is unique to that infection which allows us to differentiate someone infected with Mycobacterium tuberculosis from Streptococcus pneumoniae or other forms of bacterial and/or viral pathogens.

Referring to FIG. 10, a handheld point of care device apparatus capable of analyzing biological specimens from a subject is generally referred to by reference numeral 10. Preferably, such a handheld device 10 would weigh less than 5 kg, and still more preferably less than 2 kg. Device 10 may also be designed to be a stationary unit. The handheld device 10 has a housing 12. As shown, the handheld device 10 has a handle 14 for ease of transport, although it will be appreciated that the handle 14 may not be included. Housing 12 has a receptacle 16 for receiving a biological specimen, which is connected to a sensor 18. The device can receive a sample such as urine or blood of less than 10 mL and preferably less than 1 mL. The sensor 18 is capable of identifying a certain number of metabolites, which might, for example, be sets of 2 to 256 metabolites. Preferably, the device's sensor 18 can also determine the concentration of each of the metabolites. The sensor 18 then connects to a processor 20. As shown, sensor 18 and processor 20 are separate units, but they may also be integrated into a single unit. The processor 20 may have memory for storing results, or there may be separate memory connected to the processor 20. Processor 20 will connect to a screen 22, where screen 22 provides an interface for the user. Screen 22 will show results from the testing. In a preferred embodiment screen 22 may be a touch screen for entering input to the device, or the handheld device 10 may have a separate input device such as a keyboard or a connection to a computing device. Handheld device 10 as shown is powered with a connection to an electrical outlet using powercord 24, but it will be appreciated that other methods may be used. A preferred embodiment would be battery powered but the device may also have provision to power the device by other means such as an AC to DC converter. Such other power source, including powercord 24 could be used to recharge a battery. Alternatively to the sensor method described above, an internal standard may be provided by the handheld device 10 into the sample in the receptacle 16. The internal standard is of a known concentration and a precisely controlled volume of standard is added to a precisely controlled amount of the sample so that the concentration of the standard in the subject's specimen is known. The handheld device 10 determines the respective concentration of each of the metabolites and thereby generates a profile for an unknown state of disease. Using multivariate statistical analysis, the handheld device 10 compares the profile for the unknown state of disease to the profile of a first state of disease and a second state of a disease to determine a probability the subject has one of the first state of disease or the second state of disease. The device may be programmed to follow any of the steps described herein.

Metabolomics can differentiate Chronic from Active Disease States

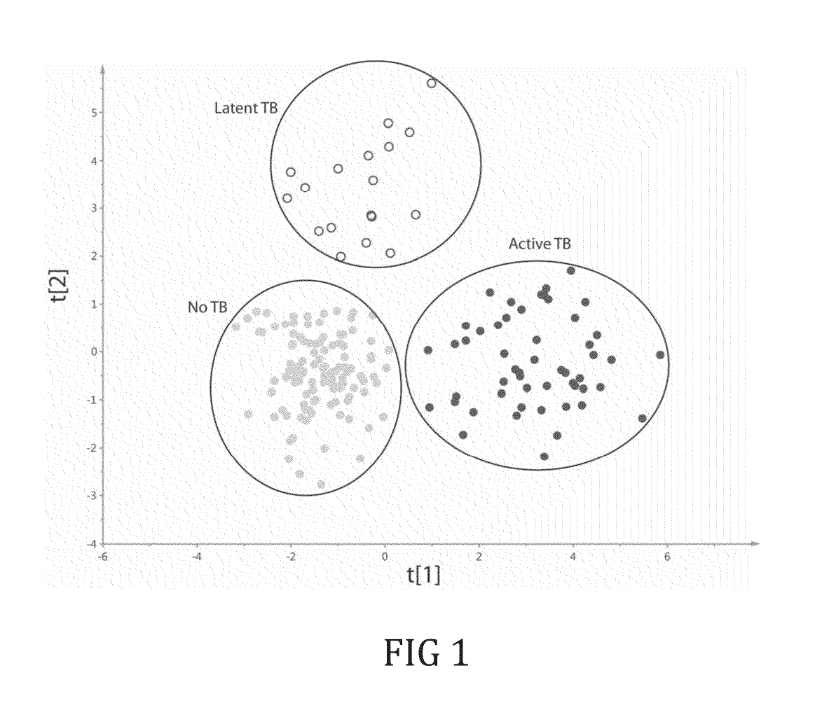

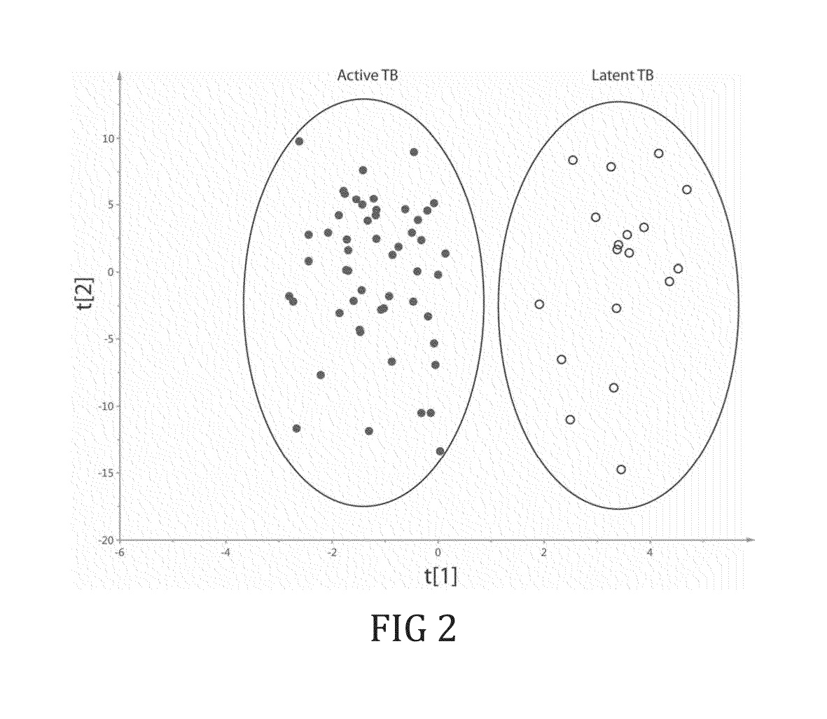

Latent tuberculosis (LTBI) is a common problem, and is the cause of approximately 80% of the active cases of tuberculosis in the United States. While LTBI does not pose an infection risk for others, it is a problem for those with HIV, cancer or other conditions in which an individual may be immunosuppressed. The ability to differentiate a latent disease state from a healthy or active disease state is important, as those in a latent disease state may be treated to prevent an active TB state. Moreover, it would be of tremendous benefit to be able to monitor the incidence of active and latent TB states within communities. Building on our earlier work on active TB, we studied a cohort of 17 individuals with various forms of latent TB. FIG. 1 shows an OPLS-DA scores plot showing two different disease states relative to control based on urinary metabolite concentrations derived from 1H NMR spectra of control subjects (no tuberculosis (n=100) (grey circles)), versus those with active TB (n=50) (black circles), and those with latent TB (n=18) (open circles). FIG. 2 shows an OPLS-DA scores plot showing two different disease states relative to each other based on urinary metabolite concentrations derived from 1H NMR spectra of individuals with active TB (n=50) (black circles), and those with latent TB (n=18) (open circles). In both cases, clear separation may be observed. These results are significant in that they illustrate the ability of metabolomics to separate chronic or latent infection states from an active infection state or a healthy state. Moreover, they illustrate that metabolomics may be used to monitor chronic or latent infection states and differentiate them from an active infection state.

Metabolomics of HIV+

On a cohort of urine samples from TB+/HIV+ and TB−/HIV+ individuals, we observed that those with HIV are differentiable from those without HIV regardless of TB status. Moreover, we were able to differentiate between those HIV+ individuals with TB from those without TB. Since this was a pilot study on a relatively small number of individuals, we sought to obtain additional samples from HIV+ individuals without the complication of TB co-infection.

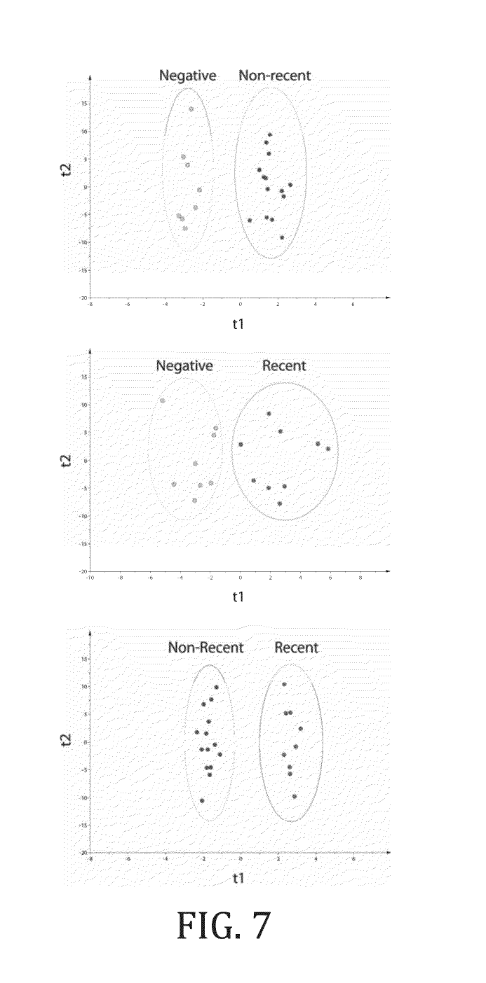

We recently completed a study comparing non-recent (ARV naïve) HIV+(−1 year to 6 years) with HIV− individuals, as well as recent (ARV naïve) HIV+(24 days to 78 days) versus non-recent HIV+ (FIG. 7). With a cohort of samples (30 total), we were able to see complete separation of recent versus non-recent infection (from CV-ANOVA, p-value 0.0072), as well as between HIV− and HIV+, confirming our earlier work with HIV.

This work indicates that an appropriate biomarker profile highly correlated to HIV recency using a urine profile may be developed. While this has many advantages, some disadvantages of using urine as a medium to detect disease state may relate to cultural differences in food and sanitation conditions in certain settings. Although our TB biomarkers were not sensitive to sanitation conditions or cultural differences in food, we cannot rule these conditions out as possible confounders for an HIV recency test. Thus, in the case that the urine profile may not be generally applicable in all settings, at the same time a marker profile using serum and plasma was developed to allow for greater flexibility in testing, and may prove more accurate.

A logical extension of this work is its applicability in other diseases such as neurological diseases (including but not limited to Parkinson's disease, Alzheimer's disease, autism), metabolic diseases (including but not limited to type 1 and type 2 diabetes, metabolic syndrome, cardiovascular disease, nonalcoholic steatohepatitis), gastrointestinal diseases (including but not limited to Crohn's disease, ulcerative colitis, irritable bowel syndrome, celiac disease), infectious diseases (as outlined herein, aerosolized, or vector-driven, and including other types of infection), cancer (including pre-cancerous states, benign states, in situ states, active states, metastatic states, and other states that may occur), and other diseases for which specific states of the disease can be delineated through specific metabolic processes that can be measured as outlined here. These diseases may be observable in either humans or animals. Additionally, diseases of the plant kingdom, such as transferred through insects, the environment, etc. may also be delineated in a similar manner.

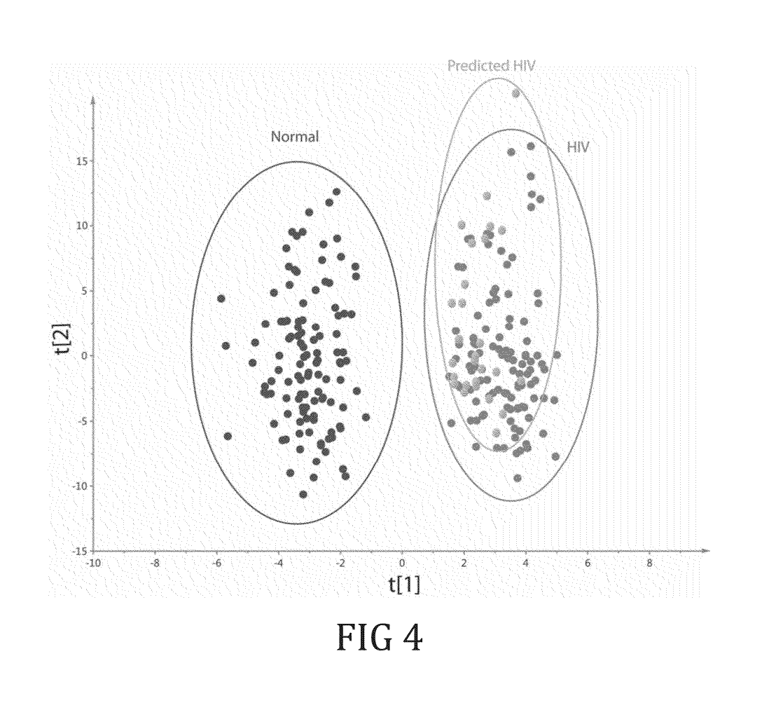

We have studied a cohort of individuals recently diagnosed with HIV to evaluate the early detection capacity of our reference profile for HIV (FIG. 4). We collected ARV-naïve urine samples from patients at a Toronto HIV clinic following a positive result on a rapid point of care test, and at three intervals thereafter (1 week, 2 weeks and 4 weeks following the point of care test) after starting ARV therapy. These sequential samples, together with the infection dates and other clinical information, allowed us to assess the urinary metabolite screen's utility as an early detector of the metabolic changes associated with HIV infection regardless of setting (third world or first world). These results demonstrate the utility and help us to more fully understand the early metabolic changes that occur with HIV infection, and provides information that is useful to aid development of useful therapeutics.

Technology and Methods:

Our method is to use nuclear magnetic resonance (NMR) spectroscopy combined with multivariate statistical analyses to develop reference metabolite profile patterns of different disease states. Comparison of profiles derived from individuals for which disease status is unknown with the reference profiles determines the health or disease state of the subject.

High-resolution NMR spectroscopy is an established analytical tool that is becoming common-place in the field of Metabolomics. This technique has proven to be advantageous over other analytical methods as it offers speed and sensitivity in identifying and quantifying literally hundreds of small molecules that vary in concentration from micromolar to molar concentrations in complex mixtures such as serum, urine, saliva, or tissue homogenates. NMR spectroscopy has several advantages over other techniques such as Mass Spectrometry (MS) or Gas Chromatography (GC). First, NMR requires very small sample volumes (0.2-0.6 mL). Second, samples require very little or no pretreatment, which allows spectra to be obtained within minutes. Third, NMR is able to obtain a complete metabolic profile of a sample without pre-selecting for compounds of interest, as it does not require the a priori separation of compounds by chromatographic methods or derivitization to facilitate detection or separation. Fourth, NMR is able to handle dynamic concentration ranges with little problem. Fifth, NMR is non-destructive, and a sample may be re-analyzed or subjected to other types of analysis once NMR acquisition is complete. Lastly, maintenance of an NMR spectrometer is minimal, requiring nitrogen and helium fills only a few times per year (in contrast to the constant need of solvents, requirement of skilled individuals to use them, maintenance and downtime issues, etc. for Mass Spectrometry analysis), and in the experience of this team has very low downtime. This enables the analysis of a wide range of compounds within a single sample. Despite the advantages of this technique, NMR has not been used extensively in the past because manual analysis of the complex spectrum requires a skilled technician and can be time consuming since a 1H NMR spectrum of a biofluid or tissue is extremely complex, consisting of thousands of well-resolved signals. However, automated pattern recognition techniques are now being used facilitate the analytical process by shortening analysis times and eliminating subjective data interpretation.

Metabolomics by NMR exploits the inherent qualitative and quantitative nature of the NMR method. Individual metabolites exhibit unique spectral signatures that are consistent and reproducible for a given set of overall sample conditions such as pH and salt concentrations, Metabolite concentrations in a given sample can be accurately determined by reference to an internal standard. With appropriate preparation of a constituent database of the estimated 1400 endogenous metabolites present at greater than 1 micromolar and intelligent software tools that model the spectrum using the database, an accurate listing of metabolites and their corresponding concentrations can be obtained. The metabolite data can then be combined with clinical and laboratory medical data and statistically analyzed to determine novel correlations between combinations of changing metabolite concentrations and observed clinical and pathogenic presentations.

How this Relates to Broader Context of Ongoing Activities in the Field

Incidence is a primary outcome measure for most HIV prevention activities, but the currently available biological assays suffer from high false recent rates, short mean duration of recency, and low suitability for field use in developing countries.

We have developed techniques for analysis of NMR spectra which permits the quantitative assessment of health states, allowing evaluation of health, disease, and/or infecting pathogen(s) simultaneously with a single sample. We are able to quantify more than 100 common metabolites in a single urine specimen concurrently and rapidly, and through the use of statistical methods have developed robust biomarkers for a number of infections and diseases, including HIV. The use of NMR is presently restricted to laboratory environments, but urine specimens may be easily collected in field settings by minimally trained individuals and transported to the lab. Once the specimens are received, the turn-around time is a few minutes by a trained individual. Once a point-of-care handheld NMR spectrometer, or other sensor technology that is capable of identifying and quantifying concentration for 100 or more metabolites at one time, is commercialized, our biomarker profile test could be completed by a minimally trained individual from collection to analysis within minutes using a point of care benchtop or handheld device.

Other methods for analysis of NMR spectra include using binning or variable binning techniques. These techniques are effective and fast for screening large populations, but are not able to effectively handle changes related to differences in diet, drug use, or differences in sample pH or ionic strength since metabolites are not specifically identified. Moreover binning, or “bins” or “buckets” or “chemical shift windows”, are likely to contain multiple metabolites and cannot identify specific metabolites or determine concentrations of discretely identified metabolites due to other factors that affect the spectral content in that range beyond the metabolites assigned to that bin, such as baseline, peak carry-over from adjacent bins, and other factors.

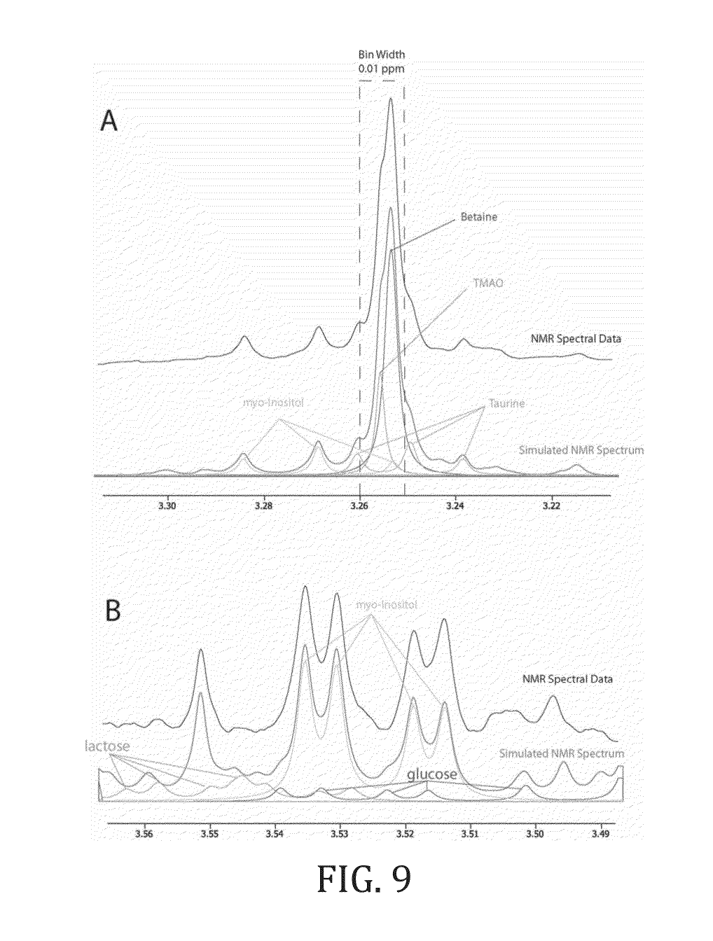

The binning approach does not account for the possibility of overlapping peaks in the spectra and that each peak may be varied by other factors other than the state being studied. Accordingly, this approach is inherently deficient and inaccurate. An example of a bin with overlapping spectra is shown in FIGS. 9a and 9b. As can be seen, the overall spectral data is affected by four separate metabolites, each of which may vary in unexpected ways.

The claimed methods allows concentrations of multiple, discretely identified metabolites to be used. This difference allows a more accurate profile to be determined, which can become very important due to the variations in the circumstances of each individual. Conditions and variations between individuals will affect the concentration of various metabolites. These conditions and variations of a patient (known or unknown) may falsely increase or decrease the signal strength in a particular bin using the binning technique. This would give a false measurement for the metabolite that is associated with that bin, and would therefore affect the conclusion to be drawn from that measurement.

By measuring concentrations of discrete metabolites, it is possible to obtain a more accurate profile and calculate a more accurate probability of the biological test sample having the diseased state. First, the reliability of the concentrations measurements is improved as compensation is made for the effects of overlapping peaks and multiple peaks. In addition, with a predetermined profile comprising a plurality of data sets, it is possible to differentiate between differences in profiles due to other conditions and those due to a disease state.

We use an internal standard to aid in the determination of concentration of discrete metabolites and it compensates for errors introduced into the determination of concentration where such errors arise because of the NMR experiment acquisition time. Short acquisition times and the absence of an internal standard leads to errors and results in an unreliable determination of concentration.

Another consideration is the so-called T1 (or spin-lattice) relaxation time. After an RF pulse, nuclear spins relax according to a time constant called T1 (which is 1/R, where R is the rate of relaxation)—which is the time for the magnetization to relax back to its equilibrium position. Each nucleus in a molecule experiences a slightly different nuclear environment, and since the nuclear environment is different, the T1 will be different. This is well known and is described in NMR textbooks (For example “Modern NMR techniques for Chemistry Research” by Derome, c1987). It is also well known to people familiar with NMR that you can identify and quantitate compounds using the frequency domain representation of the time domain data acquired during the NMR experiment. To create the frequency domain representation it is well known to use a mathematical operation called the Fourier transform. The mathematical operation can introduce an error to the area of the peaks in the resultant spectroscopic representation and this error is directly related to the amount of time domain data used to do the transform. A longer period of data in the time domain reduces the error of the mathematical operation in the frequency domain.

For small molecules, the T1 relaxation time can be large (on the order of several seconds), and it can vary (from <1 s to >20 s).

The typical 1D NMR experiment runs for 1-2 s per FID acquisition. To ensure complete relaxation so as to avoid introducing errors into the Fourier transform, it is generally understood by people familiar with NMR that you would need to run the experiment for 5 times the maximum T1, and this would allow you to reliably determine quantitation with an acceptable amount of error.

Alternatively, to minimize or eliminate the error you can use an internal reference standard. By using an internal reference you can reduce the period of the time domain acquisition since the error of the transformation is a known quantity and is compensated mathematically.

We run our acquisition at 4 s and our relaxation delay is 1 s, so our total experimental time is 5 s. We do this because we created a library that was collected with those parameters.

This is not 5× the longest T1 in the sample, but since we have an internal standard that was standardized to this time, we can quantify with minimum or no error. Because of this, when we compare our reference concentration to our concentration of metabolite in the solution, we can get accurate concentration.

With binning methods—it is certain that the area under a peak in the spectrum does not correspond to accurate concentration since the acquisition time is generally kept short and is thus not 5× the longest T1 in the sample. Therefore, most molecules will not be able to be quantified because of the large error associated with the mathematical transform. Thus binning techniques will allow comparison of spectra, but do not obtain a reliable indication of concentration (“absolute concentration”).

### EXAMPLES

Public health users of incidence assays refer to recent HIV infection as HIV infections likely to have occurred within 6 to 12 months, and which may or may not be acute infections (first two months). Duration of recency is key to accurate incidence measurement, but unreasonably large sample sizes are needed for cross sectional surveys using current serologic assays. We anticipate that using 1H NMR-based metabolite analysis, we will be able to classify unknown samples as recent or non-recent with high confidence. (FRR <5%, and MDRI of 4 months).

It will be understood that the method steps described in each of the examples below may be generalized and used with respect to developing and comparing other profiles for different patients and different diseases or disease states.

### Example 1

### Define a Biomarker Profile for Recency

We differentiated recent (<6 months from infection) from non-recent (>1 year from known infection) using well characterized specimens obtained from ARV naïve individuals provided to us in an unblinded fashion (FIG. 7). Using a cohort of approximately 30 individuals (8 HIV−, 9 recent HIV+, 14 non-recent HIV+), we developed a proof of concept HIV recency biomarker that allowed differentiation between HIV− and both recent (<3 months) and non-recent (>1 year) HIV+, as well as recent HIV+ from non-recent HIV+using urinary metabolite profiling. In this set of data, our results were considered significant (p=0.0072), providing a power of 77%. To confirm these results in urine, increase the power of our biomarker set, and to correlate and determine the profile in serum, we analyzed additional specimens of urine and developed profiles for urine and plasma from well-characterized specimens. The specimens consisted of urine and plasma from HIV+ recent infections (<6 months), HIV+ non-recent infections (>12 months), both ARV-naïve, and HIV− samples. All samples were completely clinically characterized, which allowed us to determine if any of the samples may have other co-morbidities that may affect the efficacy of our biomarker determination. The number of subjects required for our study was based on our experience in performing metabolomics studies together with our previous study on tuberculosis described earlier.

Experimental Method:

Specimen collection: Specimens of urine (1 mL) and serum (1 mL) were provided and were shipped on dry ice, and once received, stored in a −80° C. freezer until analysis.

NMR Analysis: Urine samples were prepared for NMR analysis by completely thawing at room temperature, followed by centrifugation to remove particulate matter. To 585 μL of urine supernatant, 65 μL of the internal standard consisting of 5 mM DSS-d6 (2,2,3,3,4,4-d6-3-(trimethylsilyl)-1-propane sulfonic acid), 0.2% sodium azide (NaN3), in 99.9% deuterium oxide (D2O) was added. The resonant frequency of DSS-d6 allows it to act as a chemical shift reference so metabolite peaks may be identified based on their resonant frequency (compared to DSS-d6). The area under the DSS-d6 peak (at 0 ppm), which corresponds to a known concentration, acts as a reference with which to compare the integrals of the unknown peaks from metabolites in the spectrum to obtain quantitative concentration information. Sodium azide acts as a preservative by preventing bacterial growth in the urine while the sample is being prepared and during data acquisition. Deuterium oxide is required as the NMR spectrometer field frequency lock (to ensure that the field does not drift while the spectrum is being acquired). Sample pH was adjusted to approximately 6.8 by the addition of small amounts of NaOH or HCl. 600 μL of sample was placed into a 5 mm NMR tube as described in. Data was acquired using an Agilent 600 MHz NMR spectrometer equipped with a triaxgradient 6 mm HCN probe using a NOESY pulse sequence with water saturation of 0.9 s during the 1.0 s prescan delay, a mixing time of 100 ms, 12 ppm sweepwidth, 4 s acquisition time, 8 dummy scans, and 32 transients. All spectra were zero-filled to 128 k data points, and a weighted Fourier transform with 0.5 Hz line-broadening was applied with manual phase and baseline correction. Metabolite profiles were derived from targeted profiling analysis using NMRSuite v6.1 Profiler (Chenomx, Inc.) as described. Briefly, profiler is linked to a database of approximately 300 metabolite molecules whose unique NMR spectral signatures are encoded. Comparison of NMR spectra, derived from urine with a spectral signature library results in a list of compounds together with their respective concentrations. All compounds in the database have been verified against known concentrations of reference NMR spectra of the pure compounds and have been shown to be reproducible and accurate.

Plasma samples were prepared for NMR analysis by thawing 1 mL of sample, and filtering through Amicon 3,000 MW cut-off filters by spinning at 7,500×g for 30 min at 4° C. To 585 μL of serum filtrate, 65 μL of the internal standard DSS-d6 (at 5 mM) was added as described above. Sample pH was adjusted to approximately 6.8 by the addition of small amounts of NaOH or HCl, and 600 μL of sample was placed into a 5 mm NMR tube as described above. Samples were run and spectra was analyzed as described above for urine.

Statistical Analysis: After generation of NMR data, results were analyzed using multivariate statistical analysis implemented in Simca-P+11 including principal components analysis (PCA), partial-least squares analysis (PLS-DA) and orthogonal PLS-DA (OPLS-DA) to determine if there were any common differences in metabolite profiles. PLS-DA and OPLS-DA are regression extensions of PCA, an unsupervised multivariate statistical analysis method that is essentially a dimensionality reduction technique, reducing multidimensional data into coherent subsets that are independent of one another, allowing identification of those variables that are interrelated. PLS-DA and OPLS-DA both use class information to maximize the separation between observations (recent HIV+ or non-recent HIV+). The variable importance in the projection (VIP) value of each metabolite was calculated to indicate its contribution to the classification of the samples. The quality of the models was judged by the goodness-of-fit parameter (R2) and the predictive ability parameter (Q2), which is calculated by an internal cross-validation of the data and the predictability calculated on a leave-out basis, as well as by CV-ANOVA. Upon identification of significant metabolite variables, univariate statistical analyses were performed to confirm between-group differences. Metabolites were tested for normality, and all variables that were not normally distributed were transformed to reflect a normal distribution. Group means were compared using t-tests. When transformation failed to achieve normality, group means were compared using the Wilcoxon rank-sum tests. Significance was set at α=0.05.

### Example 2

### Evaluate False Recent Rate and Mean Duration of Recency

False recent rate (FRR) and mean duration of recency (MDRI) are two important characteristics that need to be optimized for generation of an effective HIV incidence assay. The ideal assay would have a minimal FRR (preferably less than 2%, but required to be less than 5%), with an MDRI of at least 4 months, but preferably 6-12 months.

Description and Assumptions:

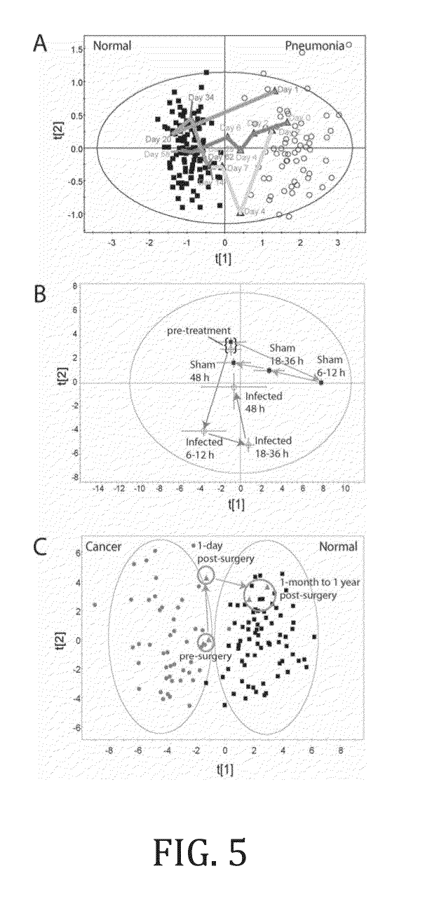

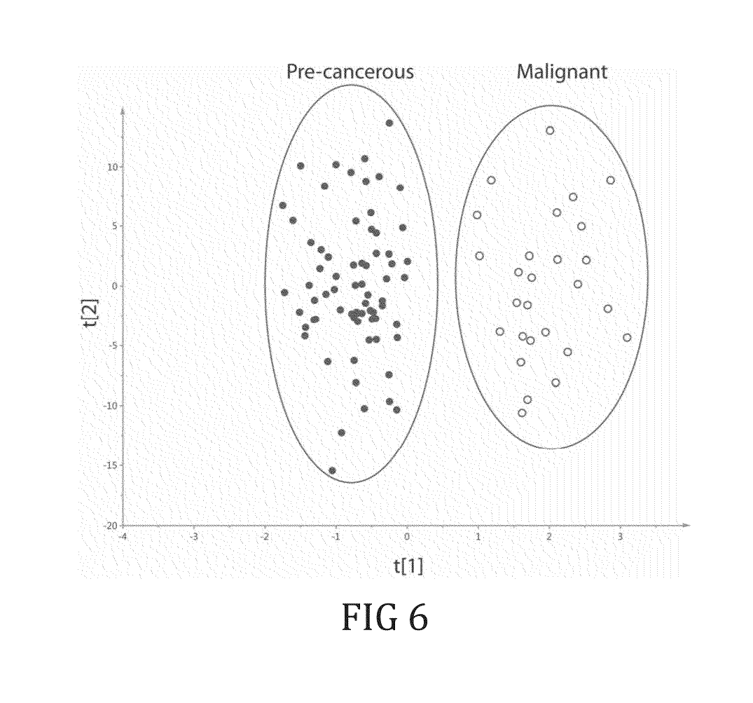

Previous published (in pneumonia and IBD) and unpublished work (in rabies and cancer) show that we can observe changes from one metabolic state to another occurring over a period of time (FIG. 5). We are able to model the metabolic changes as individuals convert from acute infection to chronic infection, and are able to use these trajectories to estimate the duration of a disease state such as recency.

Experimental Method:

Statistical Analysis: After analysis of samples (described above), we determined the sensitivity, specificity, False Recent Rate (“FRR”) and Mean Duration of Recent Infection (“MDRI”) of both the urinary and plasma and serum metabolomics HIV recency assays. To determine the FRR we predicted recent and non-recent by predicting class on the basis of the previous model built. This model provided a baseline for evaluating false recent rate with respect to ARV-treated HIV+, long term survivors and AIDS state. We conducted a general/preliminary analysis and determined the proportion of recent results since time of infection, and followed the metabolic trajectories as the patients converted from acute to chronic infection. We then interpolated/curve fit to individual series to obtain a distribution for time in “recent” infection per subject.

Results Measurement: We were able to determine the FRR and MDRI using our metabolite biomarker pattern. The qualification was done in two steps so that were able to refine our biomarker profile.

### Example 3

### Distinguish between ARV-Treated, ARV-naïve, and HIV−

### Description and Assumptions:

Since false recency may be associated with the viral suppression of antiretroviral (ARV) use, we propose to determine whether those that have been treated with ARV are metabolically distinguishable from those that are ARV naïve or HIV−. We expect the biomarker profile for recent infection (ARV-naive) will be distinguishable from the biomarker profiles for ARV treated/suppressed.

Experimental Method:

Statistical Analysis: To determine whether ARV-treated patients that are >12 months post-infection will classify as recent, spectral data for ARV-treated/virally suppressed HIV+ individuals that are more than 12 months from time of infection are compared with our ARV-naïve and HIV− subjects, samples analyzed as described above, and evaluated for classification by predicting class on the basis of the model built from the developmental set. It is expected that metabolite analysis will identify those treated with ARV drugs from those who are medication naïve since the individuals on medication will produce distinct disease state from an individual who is medically naïve.

### Example 4

### Distinguish AIDS State of Disease

Description and assumptions: Specimens collected at the AIDS state of HIV infection will be used, and NMR spectra will be generated. We will determine the profile of individuals with AIDS and expect to differentiate them from recent, healthy and ARV-naïve since the AIDS state of disease is different from the recent state and the chronic state of disease.

Experimental Method:

Statistical Analysis: Spectral data from AIDS patients will be compared with recent and non-recent HIV+ as well as HIV− cohorts. These results will be important to understand the states of disease (recent, chronic disease, and end-stage disease), and additionally help to understand elite controllers.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

The scope of the following claims should not be limited by the preferred embodiments set forth in the examples above and in the drawings, but should be given the broadest interpretation consistent with the description as a whole.

1. A method of determining the state of a disease in a subject, the method comprising the steps of:
obtaining at least a first set of diseased biological samples from subjects known to have a first state of the disease and a second set of diseased biological samples from subjects known to have a second state of the disease, each biological sample comprising a plurality of biomarkers, wherein the first state of the disease and the second state of the disease are differentiated by a predetermined period of time and wherein the first state of disease and the second state of disease are states of cancer, and are selected from preclinical, indolent, metastatic, aggressive, and remission;
for each sample, measuring the concentration of a set of biomarkers from among the plurality of biomarkers and generating a profile for each state of the disease, each profile comprising the concentrations from a plurality of samples;
obtaining a biological sample from a subject having an unknown state of disease and measuring the concentration of a set of biomarkers from among the plurality of biomarkers and generating a profile for the unknown state of disease; and
comparing the profile for the unknown state of disease to at least the profile of the first state of disease and the second state of disease to determine whether the subject has one of the first state of disease or the second state of disease.

2. The method of claim 1, wherein the disease is HIV infection.

3. The method of claim 1, wherein the first state of the disease is a recent infection and the second state of the disease is a chronic infection.

4. The method of claim 3, wherein the recent infection may be an infection contracted within a period of less than 6 months and the chronic infection may be an infection contracted in a period of over 12 months.

5. The method of claim 1, further comprising at least a third state of disease.

6. The method of claim 5, wherein the third state of the disease comprises no disease.

7. The method as claimed in claim 1, further comprising the step of developing a prognosis of the subject based on the comparison of the profiles.

8. The method of claim 1, wherein the biological sample is one of blood, blood plasma, blood serum, cerebrospinal fluid, bile acid, saliva, synovial fluid, pleural fluid, pericardial fluid, peritoneal fluid, feces, nasal fluid, ocular fluid, intracellular fluid, intercellular fluid, lymph fluid, and urine.

9. The method of claim 1, wherein comparing the profile comprises using multivariate statistical analysis.

10. The method of claim 9, wherein the multivariate statistical analysis is selected from the group consisting of principal component analysis, discriminant analysis, principal component analysis with discriminant analysis, partial least squares, partial least squares with discriminant analysis, canonical correlation, kernel principal component analysis, non-linear principal component analysis, factor analysis, multidimensional scaling, and cluster analysis.

11. The method of claim 1, wherein each profile comprises information derived from a respective region of a score plot using multivariate statistical analysis.

12. The method of claim 1, wherein the respective concentration of each of the identified metabolites is determined using a spectrometric technique, wherein the spectrometric technique is any one of liquid chromatography, gas chromatography, liquid chromatography—mass spectrometry, gas chromatography—mass spectrometry, high performance liquid chromatography—mass spectrometry, capillary electrophoresis—mass spectrometry, nuclear magnetic resonance spectrometry (NMR), raman spectroscopy, and infrared spectroscopy.

13. (canceled)

14. A method of profiling the recency of a disease, comprising the steps of:
obtaining at least a first set of diseased biological samples from subjects known to have a first state of the disease and a second set of diseased biological samples from subjects known to have a second state of the disease, each biological sample comprising a plurality of biomarkers, wherein the first state of the disease and the second state of the disease are differentiated by a predetermined period of time; and
for each sample, measuring the concentration of a set of biomarkers from among the plurality of biomarkers; and
using multivariate statistical analysis, generating a profile for each state of the disease, each profile comprising the concentrations from a plurality of samples.

15. The method of claim 14, wherein the disease is HIV infection.

16. The method of claim 14, wherein the first state of the disease is a recent infection and the second state of the disease is a chronic infection.

17. The method of claim 16, wherein the recent infection may be an infection contracted within a period of less than 6 months and the chronic infection may be an infection contracted in a period of over 12 months.

18. The method of claim 14, further comprising at least a third state of disease.

19. The method of claim 18, wherein the third state of the disease comprises no disease.

20. The method of claim 14, wherein the biological sample is one of blood, blood plasma, blood serum, cerebrospinal fluid, bile acid, saliva, synovial fluid, pleural fluid, pericardial fluid, peritoneal fluid, feces, nasal fluid, ocular fluid, intracellular fluid, intercellular fluid, lymph fluid, and urine.

21. The method of claim 14, wherein the multivariate statistical analysis is selected from the group consisting of principal component analysis, discriminant analysis, principal component analysis with discriminant analysis, partial least squares, partial least squares with discriminant analysis, canonical correlation, kernel principal component analysis, non-linear principal component analysis, factor analysis, multidimensional scaling, and cluster analysis.

22. The method of claim 14, wherein each profile comprises information derived from a respective region of a score plot using multivariate statistical analysis.

23. The method of claim 14, wherein the respective concentration of each of the identified metabolites is determined using a spectrometric technique, wherein the spectrometric technique is any one of liquid chromatography, gas chromatography, liquid chromatography - mass spectrometry, gas chromatography - mass spectrometry, high performance liquid chromatography - mass spectrometry, capillary electrophoresis - mass spectrometry, nuclear magnetic resonance spectrometry (NMR), raman spectroscopy, and infrared spectroscopy.

24-26. (canceled)

27. A method of determining the state of a disease in a subject, the method comprising the steps of:
obtaining a plurality of sets of diseased biological samples from subjects, each subject having a known state of the disease, the disease comprising cancer, each biological sample comprising a plurality of biomarkers, wherein each state of the cancer being one of aggressive, active, acute, recent, chronic, indolent, non-recent, primary, persistent, remission or subclinical;
for each sample, measuring the concentration of a set of biomarkers from among the plurality of biomarkers and generating a profile for each state of the cancer, each profile comprising the concentrations from a plurality of samples from subject having the same state of the cancer;
obtaining a biological sample from a subject having an unknown state of the cancer and measuring the concentration of a set of biomarkers from among the plurality of biomarkers and generating a profile for the unknown state of the cancer; and
comparing the profile for the unknown state of the cancer to the profile of at least two states of the cancer determine whether the subject has one of the states of the cancer.
